(19) United States
(12) Patent Application Publication
(10) Pub. No.: US 2005/0009772 A1
(43) Pub. Date: Jan. 13, 2005

(54) Methods and compositions for the treatment of glaucoma and other retinal diseases

(75) Inventor: Joseph Caprioli, (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(73) Assignee: The Regents of the University of California

(21) Appl. No.: 10/841,207

(22) Filed: May 6, 2004

Related U.S. Application Data
(60) Provisional application No. 60/468,554, filed on May 6, 2003.

(51) Int. Cl. A61K 48/00

(52) U.S. Cl. 514/44

(57) ABSTRACT

Disclosed herein are methods and compositions for treating glaucoma and other disorders related to degeneration of retinal neuronal cells, by treating a subject with a composition capable of inducing or increasing the expression of the 70 kD family of heat shock proteins (HSP70) in retinal neurons. Preferred embodiments include geranylgeranylacetone and/or gene therapy applications.

### CROSS-REFERENCE TO RELATED APPLICATION(S)

This application is claims priority to U.S. Provisional Patent Application Ser. No. 60/468,554, filed May 6, 2003. The entire disclosure of the prior application is considered to be part of the disclosure of the instant application and is hereby incorporated by reference herein.

### TECHNICAL FIELD

This invention relates generally to the field of prevention of retinal neuronal cell degeneration, and more specifically to methods for treating glaucoma, macular degeneration and other neurodegenerative retinal diseases by inducing expression of HSP70 proteins utilizing geranylgeranylacetone and gene therapy.

### BACKGROUND OF THE INVENTION

Glaucoma is the second-leading cause of blindness in the United States behind macular degeneration, a degenerative disease of the central retina in the elderly. Glaucoma is characterized by progressive optic nerve damage with selective loss of retinal ganglion cells (RGCs). Quigley et al., Opthalmology 95:357-63 (1988); Sommer et al., Arch. Opthalmol. 109:77-83 (1991); Glovinsky et al., Invest. Ophtalmol. Vis. Sci. 32:484-91 (1991). Reduction of intraocular pressure, the standard treatment for glaucoma, is only partially protective against retinal damage.

The present inventors have previously demonstrated that protection against neuronal degeneration can be mediated by induction of the stress response in retinal neuronal cells. Park et al., Invest. Opthalmol. Vis. Sci. 42:1522-1530 (2001); Caprioli et al., Invest. Opthalmol. Vis. Sci. 37:2376-81 (1996). In particular, induction of heat shock protein 72 (HSP72) via heat stress and zinc administration was shown to have a neuroprotective effect in a rat glaucoma model, and the induction of HSP72 correlated with and increased the survival rate of RGCs in rats with elevated intraocular pressure (IOP). Park et al., supra. Unfortunately, however, heat stress is impractical for treatment of glaucoma in humans. Likewise, treatment with quantities of zinc sufficient to induce HSP72 production in human RGCs would likely lead to toxic side effects.

Significantly, although a variety of agents have been described in the art as having the ability to induce a heat shock protein response in neuronal cells in general, and in retinal neurons in particular, the ability of these agents to ameliorate the damage to retinal ganglion cells caused by glaucoma cannot necessarily be inferred. For example, 2-deoxy-D-gluycose (2DG) has been shown to protect both cerebral neurons and retinal neurons against excitotoxicity , i.e., neuronal death caused by excessive neurotransmitters, possibly through induction of HSP72. Lan et al., NeuroReport 14:2369-72 (2003). In the hands of the, present inventors, however, 2DG administration was ineffective in protecting retinal ganglion cells in the animal model of glaucoma employed herein. Thus, confirmation of efficacy in a relevant animal model is required before any actual conclusions can be drawn.

What is needed are improved compositions capable of mediating a neuroprotective effect in the retina. Ideally, such compositions would be non-toxic at therapeutic levels and bioavailable across the blood brain barrier. Still more preferably, such compositions would be orally administrable.

### RELEVANT LITERATURE

Geranylgeranylacetone (GGA), an acyclic polyisoprenoid developed and used clinically in Japan for the treatment of ulcers, protects gastric mucosa without affecting gastric acid or pepsin secretion. Murakami et al., Arzneimittelforschung 31:799-804 (1981). This cytoprotective effect has been correlated with the expression of HSPs in gastric mucosal cells induced by the systemic administration of GGA. Hirakawa et al. Gastroenterology 111:345-357 (1996); Takahashi et al. J Physiol Pharmacol. 47:433-441 (1996; Tsutsumi et al. Biol Pharm Bull. 22:886-887 (1999); Mizushima et al. Dig Dis Sci. 44:510-514 (1999); Rokutan et al. J Gastroenterol. 35:673-681 (2000).

GGA induces the expression of HSP60, HSP70 and HSP90 in gastric mucosal cells in vivo and in vitro by activating heat shock factor-1 (HSF1), the transcription factor for HSPs. Hirakawa et al., supra. It has been reported that GGA induces HSPs in numerous tissues of rats including small intestine, liver, lung, kidney and heart. Tsuruma et al. Transplant Proc. 32:1631-1633 (2000); Tsuruma et al. Transplantation Proc. 31:572-573 (1999); Yamagami et al. J Lab Clin Med. 135:465-475 (2000); Fudaba et al. Transplantation 72:184-189 (2001); Ikeyama et al. J Hepatol. 35:53-61 (2001); Ooie et al. Circulation 104:1837-1843 (2001).

Application of GGA has been proposed to have potential therapeutic benefits for treatment and prevention of ischemia/reperfusion injury, trauma, inflammation, infection, stress ulcer and organ transplantation. Rokutan et al. J Med Invest. 44:137-147 (1998). Although its potential use in neuroprotection has been proposed, see Park et al, supra, the effects of GGA in neuronal tissue or retinal neuron cells in particular have never been investigated, and its efficacy in a relevant animal model of retinal degeneration has never been proven.

### SUMMARY OF THE INVENTION

The present invention solves the aforementioned problems through the provision of therapeutic formulations comprising geranylgeranylacetone (GGA) to induce the expression of heat shock proteins, preferably HSP70 proteins, and HSP72 in particular, in retinal neurons and particularly in retinal ganglion cells. GGA is demonstrated herein to induce heat shock protein expression in RGCs whether administered orally or intraperitoneally, and to provide neuroprotective effects in a relevant animal model of glaucoma.

In one aspect, methods for inhibiting retinal degeneration in a patient suffering from a neurodegenerative retinal disease are provided, comprising the administration of a therapeutically effective amount of GGA to the patient. As evidenced herein, the therapeutically effective amount is sufficient to induce the expression of HSP70 proteins, and HSP72 in particular, and results in a neuroprotective effect on retinal ganglion cells. Retinal diseases which may be advantageously treated using the subject compositions and methods include glaucoma, macular degenerations, diabetic retinopathy, retinal vein occlusion, retinal aretery occlusion, hereditary degenerations of the retina, vaso-occlusive diseases of the retina and retinal infections.

In a preferred embodiment, methods for treating glaucoma are provided, comprising the administration of a neuroprotective amount of GGA to a patient suffering from glaucoma. In a particularly preferred embodiment, administration of the GGA is accomplished orally.

In another aspect, the invention provides methods and compositions for increasing expression of HSP70 proteins in vivo to inhibit neurodegeneration in a patient, by contacting a retinal neuron of the patient with a nucleic acid encoding a HSP70 protein. In a preferred embodiment, the retinal neuron is a retinal ganglion cell. In a particularly preferred embodiment, a patient is suffering from glaucoma.

### BRIEF DESCRIPTION OF THE DRAWINGS

FIG. 1 shows a sequence of a rat HSP72 polypeptide (SEQ.ID NO: 1) useful in one aspect of the invention.

FIG. 2 shows a sequence of a human HSP72 polypeptide (SEQ.ID NO: 2) useful in one aspect of the invention.

FIG. 3 shows a western blot analysis (A) for HSP72 (upper panel) and HSC70 (lower panel) illustrating increased HSP72 expression in RGCs after 3 and 7 days of GGA administration, but no change in HSC70 expression. There was no change in the immunoreactive band of HSP72 after administration of GGA with quercetin (Q) (4 mg/kg daily) for 7 days. Immunohistochemical staining for HSP72 showed mild immunoreactivity in RGCL cells of vehicle-treated retina (B) and an increased immunoreactivity (arrowheads) in RGCL cells of retina treated with GGA for 7 days (C). Immunohistochemical staining for HSC70 showed strong immunoreactivity in RGCL cells of vehicle-treated retina (D) and GGA-treated retina (E).

FIG. 4 shows the IOP course in each group for Experiment 2. There was a significant increase of IOP in all groups with trabecular laser photocoagulation (*P=0.001) when compared with groups without photocoagulation. Administration of GGA, vehicle or GGA with quercetin did not cause a significant change in IOP. Laser, trabecular laser photocoagulation after intracameral ink injection; GGA, GGA injection; Q, quercetin injection. Data are expressed as mean ±SEM.

FIG. 5 shows an analysis of RGCs labeled with DTMR after 5 weeks of IOP elevation. Representative micrographs of vehicle-treated control retina (A), elevated IOP retina with vehicle (B), elevated IOP retina with administration of GGA (C), elevated IOP retina with administration of GGA and quercetin (D), control (normal IOP) retina with administration of GGA (E), control (normal IOP) retina with administration of GGA and quercetin (F) were shown. Counting of DTMR labeled RGCs (G) revealed a statistically significant decrease in density of RGCs in elevated IOP retinas with administration of vehicle (*P=0.003), and administration of GGA and quercetin (†P=0.002). Administration of GGA caused a higher density in elevated IOP retina than administration of vehicle (‡P=0.048) or GGA and quercetin in elevated IOP retina (§P=0.002). GGA, GGA injection; Q, quercetin injection. Data are expressed as mean ±SEM.

FIG. 6 shows representative micrographs that illustrate optic nerve cross section for the vehicle-treated control, with a grade of I (A) and degeneration in the optic nerve section of a laser-treated eye after 5 weeks of IOP elevation showing focal degenerating axons, with an injury grade of 2 (B). Optic nerve injury grading (C) and cell counting in the RGCL (D) showed significant axonal damage and reduction of cells in the RGCL after 5 weeks of IOP elevation when compared with vehicle- or GGA-treated controls (*P <0.05). This axonal damage and reduction of cells in the RGCL was inhibited by administration of GGA (†P<0.05). GGA, GGA injection; Q, quercetin injection. Data are expressed as mean ±SEM.

FIG. 7 shows TUNEL staining of vehicle-treated control retina (A) and the retinas of laser-treated eye (B). (C) shows quantitative analysis of TUNEL positive cells in the RGCL showed a significant increase of TUNEL positive cells in all elevated IOP eyes when compared with vehicle control groups (*P=0.026). The number of TUNEL positive cells in elevated IOP retinas was reduced by administration of GGA (†P=0.02) but the reduction was reversed by coadministration with quercetin (‡P=0.017; compared with elevated IOP retina with administration of GGA). GGA, GGA injection; Q, quercetin injection. Data are expressed as mean ±SEM.

FIG. 8 shows quantitative analysis of the immunoreactive intensities of HSP72 (A) and HSC70 (B) in the RGCL after 1 week of IOP elevation. (A) Increased immunoreactivity of HSP72 was noted in RGCL cells of eyes with IOP elevation (*P=0.01) and control eyes with administration of GGA (†P=0.005) when compared to vehicle-treated eyes. Administration of GGA apparently further increased immunoreactivity of HSP72 in RGCL of eyes with IOP elevation ({P=<0.001 compared with vehicle control) but there was no statistical significance when compared with IOP-elevated eyes alone. The increase was abolished by co-administration with quercetin (§P=0.002). Increased immunoreactivity of HSP72 in control (normal IOP) retina treated with GGA was also diminished by co-administration of quercetin. (B) No change in HSC70 immunoreactivity was shown among the groups. GGA, GGA injection; Q, quercetin injection. Data are expressed as mean ±SEM.

### DETAILED DESCRIPTION OF PREFERRED EMBODIMENTS

In the foregoing Background section and in the Detailed Description that follows, citation is made to various references in the text or bibliography, which may aid one of ordinary skill in the art in the practice of the methods of the invention or in obtaining a better understanding thereof. Accordingly, each such reference cited is incorporated herein by reference to the extent necessary to aid one of ordinary skill in the art to understand practice the methods and make the compositions of the invention.

The present invention is based on the discovery that GGA is able to induce expression of HSP70 proteins in RGCs and, unlike failed candidate agents such as 2DG, is capable of mediating a neuroprotective effect on RGCs in a relevant animal model of glaucoma. Moreover, GGA is capable of mediating such effects whether administered orally or intraperitoneally, thereby demonstrating its ability to cross both the gastrointestinal membranes and the blood brain barrier to reach the retina. As evidenced herein, the induction of HSP70 proteins in RGCs by GGA provides superior benefits in comparison with prior art compounds and protocols with respect to increased efficacy and reduced toxicity.

One aspect of present invention therefore provides for methods of treating a subject to protect against degeneration of retinal neurons, and in particular embodiments, to protect against the degeneration of RGCs in a subject having glaucoma by treating the subject with GGA to induce expression of HSP70 proteins in the RGCs.

Another aspect of the invention provides for methods of protecting against degeneration of retinal neurons, particularly RGCs, by contacting a retinal neuronal cell with a nucleic acid operably configured to increase expression of HSP70 proteins in the cell. In a particularly preferred embodiment, the HSP protein is HSP72.

The terms “induced expression”, “increase expression” and grammatical variants of the same, refer to expressing HSP70 protein in a cell as a result of treating a subject or contacting a cell with a substance that causes the cell to express HSP70 to a higher degree than the cell would normally express the HSP70 if the subjected were not treated or the cell was not contacted with the substance. Measurement of the amount of HSP70 in cells may be done according to a variety of methods known in the art, including, but no limited to the immunological methods described herein. Accordingly, in various exemplary embodiments, “induced expression” refers to increased expression as a result of treating a subject animal with GGC or by contacting a cell in the animal (or a culture) with a nucleic acid operably configured to express HSP70 in the cell.

As used herein, the term “HSP70 protein” refers to any member of the heat shock protein 70 kD family, which includes, but is not limited to heat shock protein 8 (Hspa8), heat shock protein 5 (Hspa5), heat shock protein HST70 or 2 (Hspt70), heat shock protein 1A (Hspa1a or HSP72), and heat shock protein 4 (Hspa4). As with many heat shock proteins, there is a high degree of interspecies homology as shown in the following table of accession numbers:

NM_{—}024351=Rattus norvegicus Heat Shock Protein 8 (Hspa8), mRNA

NP_{—}077327=rat protein
M19141=mouse mRNA; 95% identity
AAA37869=mouse protein 8; 99% identity
BC016660=human mRNA; 89% identity
AAH16660=human protein 8; 99% identity
   NM_{—}013083=Rattus norvegicus Heat Shock 70 kD Protein 5 (Hspa5), mRNA
NP_{—}037215=rat protein
BC050927=mouse heat shock 70 kD protein 5 mRNA; 95% identity
AAH50927=mouse heat shock 70 kD protein 5; 99% identity
BC020235=human heat shock 70 kDa protein 5 mRNA; 90% identity
AAH20235=human heat shock 70 kDa protein 5; 98% identity
   X15705=Rattus norvegicus 70 kDa Heat Shock Protein HST70
CAA33735=rat protein
BC004714=mouse mRNA; 96% identity through cds
AAH04714=mouse heat shock protein 2 ; 99% identity
L26336=human Homo sapiens heat shock protein (HSPA2) gene, complete cds; 91% identity through most of cds (last 40 nucleotides of cds excluded)
AAA52698=human heat shock protein; 98% identity
   NM_{—}031971=Rattus norvegicus Heat Shock 70 kD Protein 1A (Hspala), mRNA
NP_{—}114177=rat protein
X77207=R.norvegicus Hsp70-1 gene; 99% identity to NM_{—}031971
CAA54422=rat protein; 99% identity (one amino dif) to NP_{—}114177
M35021=mouse heat shock protein 70.1 (hsp70.1) gene, complete cds; 95% identity
AAA37864=mouse protein; 98% identity
BC002453=Homo sapiens heat shock 70 kDa protein 1A, mRNA ; 92% identity over most of cds (excluding first 15 nucleotides)
AAH02453=human heat shock 70 kDa protein 1A; 96% identity
   NM_{—}153629=Rattus norvegicus Heat Shock 70 kDa Protein 4 (Hspa4), mRNA
NP_{—}705893=rat protein
BC003770=Mus musculus heat shock protein 4, mRNA; 95% identity
AAH03770=mouse protein; 99% identity
NM_{—}002154=Homo sapiens heat shock 70 kDa protein 4 (HSPA4), transcript variant 1, mRNA; 90% identity
NP_{—}002145=human protein; 95% identity

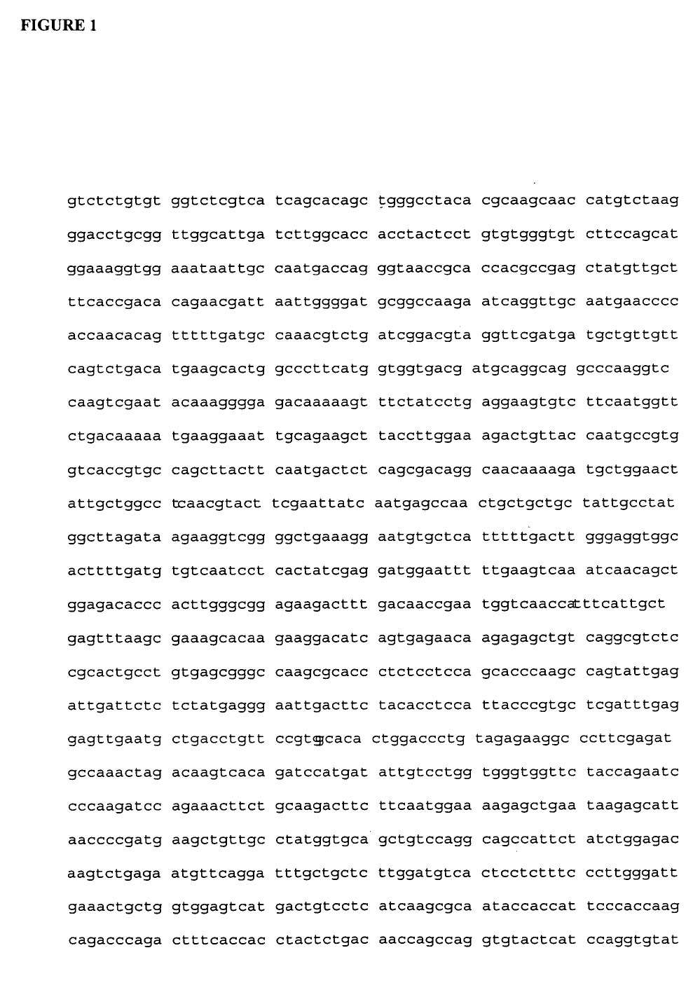

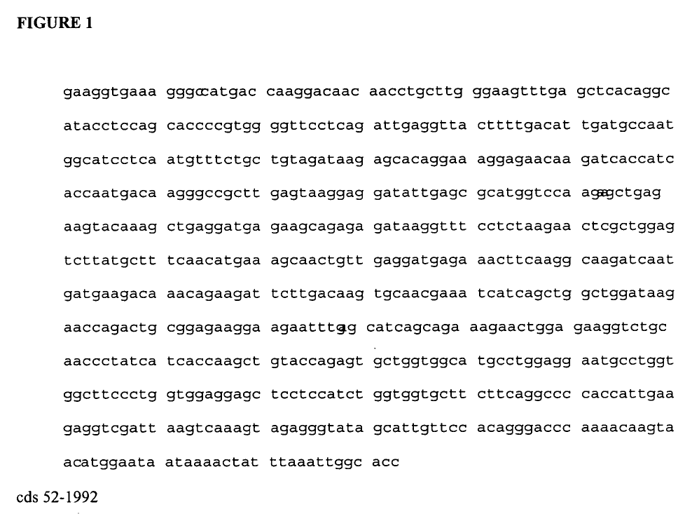

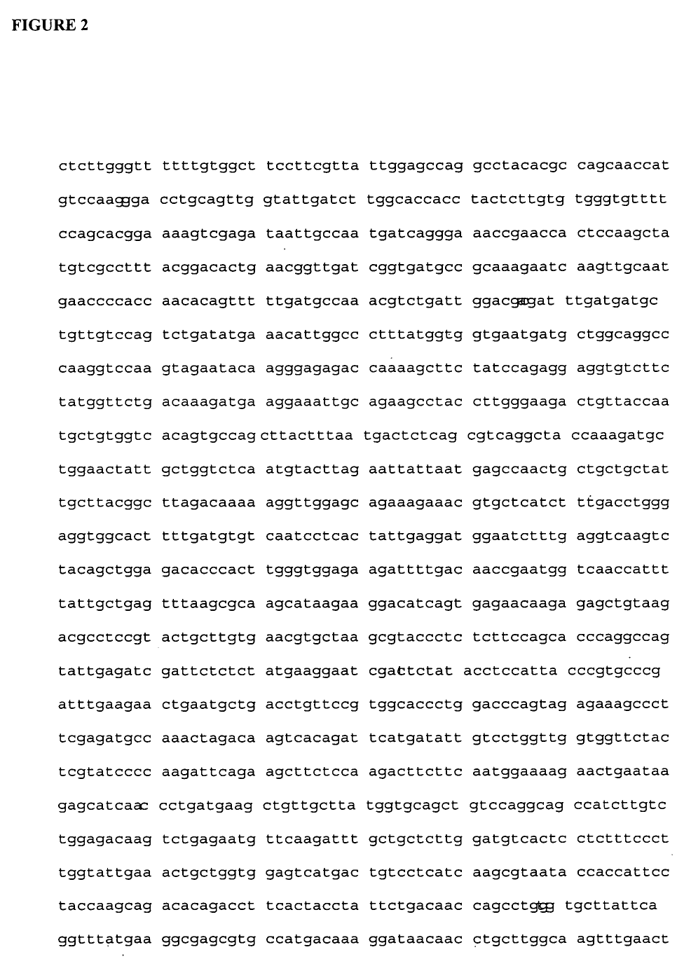

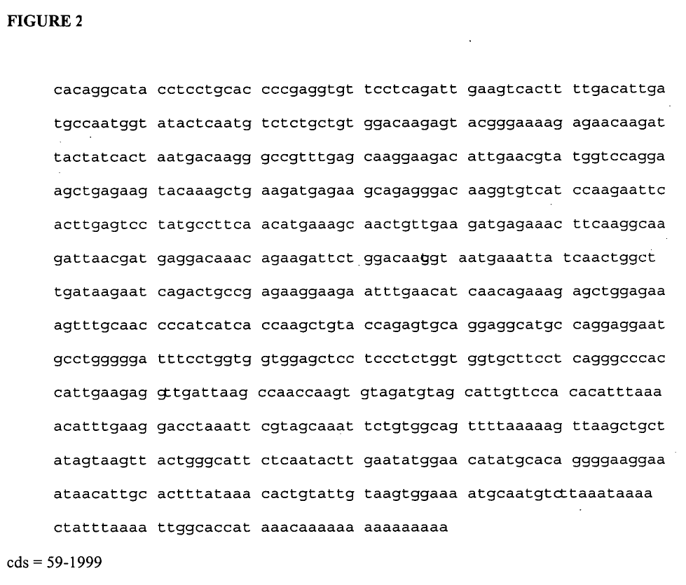

In preferred embodiments, the HSP70 protein is HSP72. In preferred embodiments, the HSP70 protein is a polypeptide encoded by SEQ.ID NOs: 1 or 2 shown in FIGS. 1 and 2, respectively.

HSP70 proteins may also include homologous polypeptides, which in various embodiments have at least about 80% sequence identity, usually at least about 85% sequence identity, preferably at least about 90% sequence identity, more preferably at least about 95% sequence identity and most preferably at least about 98% sequence identity with the polypeptides encoded by SEQ.ID NOs: 1 or 2 and which exhibit at least one biological activity that is normally associated with the HSP70 polypeptide encoded by SEQ.ID NOs: 1 or 2. One biological activity particularly pertinent to the present invention is the ability to protect neuronal cells, and particularly RGCs from degeneration when expression of the HSP70 polypeptide is induced or increased in the neuronal cell.

As is known in the art, a number of different programs can be used to identify whether a protein or nucleic acid has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman and Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucleic Acids Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149, Alan R. Liss, Inc. (1988).

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, J. Mol. Evol. 35:351-360 (1987); the method is similar to that described by Higgins and Sharp, CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215:403-410, (1990) and Karlin et al., Proc. Natl. Acad. Sci. USA 90:5873-5787 (1993). A particularly useful BLAST program is the WU-BLAST-2 program which was obtained from Altschul et al., Methods in Enzymology 266:460-480 (1996) (available at world wide web site blast.wustl/edu/blast/kEADME.html). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A percent (%) amino acid or nucleic acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the amino acid sequence of the polypeptide encoded by SEQ.ID NOs: 1 or 2. It is understood that in one embodiment, the percentage of sequence identity will be determined based on the number of identical amino acids in relation to the total number of amino acids. Thus, for example, sequence identity of sequences shorter than that of the polypeptide encoded by (SEQ.ID NOs: 1 or 2) as discussed below, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Polypeptides having HSP70 activity may be shorter or longer than the polypeptide encoded by SEQ.ID NOs: 1 or 2. Thus, in a preferred embodiment, included within the definition of HSP70 polypeptide are portions or fragments of the polypeptide encoded by SEQ.ID NOs: 1 or 2. In one embodiment herein, fragments of the polypeptide encoded by SEQ.ID NOs: 1 or 2 are considered HSP70 polypeptides if a) they have at least the indicated sequence identity; and b) preferably have a biological activity of naturally occurring HSP70 as described herein.

In addition, as is more fully outlined below, a HSP70 polypeptide can be made longer than the polypeptide encoded by SEQ.ID NOs: 1 or 2, for example, by the addition of other fusion sequences, or the elucidation of additional coding and non-coding sequences.

The HSP70 polypeptides expressed in cells by introduction of exogenous sequences encoding the polypeptides are preferably recombinant. A “recombinant polypeptide” is a polypeptide made using recombinant techniques, i.e., through the expression of a recombinant nucleic acid as described in more detail hereafter. In a preferred embodiment, the HSP70 polypeptide of the invention is made through the expression of the polypeptide encoded by SEQ.ID NO: 1 or 2, or fragment thereof. A recombinant polypeptide is distinguished from naturally occurring protein by at least one or more characteristics. The definition includes the production of a HSP70 polypeptide from one organism in a different organism or host cell. Alternatively, the polypeptide may be made at a significantly higher concentration than is normally seen, through the use of an inducible promoter or high expression promoter, such that the polypeptide is made at increased concentration levels. Alternatively, the polypeptide may be in a form not normally found in nature, as in the addition of amino acid substitutions, insertions and deletions, as discussed below.

The concentration of GGA or nucleic acid encoding a HSP70 protein will be determined empirically in accordance with conventional procedures for the particular purpose. Generally, for therapeutic purposes the subject compositions are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms or manifestations of the disorder or disease.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider includes whether the disease is acute or chronic, relapsing or remitting phase, and the progressiveness of the disease. Determining the dosages and times of administration for a therapeutically effective amount are well within the skill of the ordinary person in the art.

For any compounds used in the present invention, therapeutically effective dose is readily determined by methods well known in the art. Information pertaining to the prior clinical use of GGA for gastric ulcers can be obtained by the skilled artisan to assist in determining appropriate dosing amounts and schedules. In addition, the toxicity and therapeutic efficacy are generally determined by cell culture assays and/or experimental animals, typically by determining a LD50 (lethal dose to 50% of the test population) and ED50 (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the prior clinical history of GGA and the detailed disclosure provided herein.

In addition to GGA administration, nucleic acid molecules (DNA or RNA) encoding HSP70 proteins may also be administered as described herein. As described above, nucleic acid molecules encoding the HSP70 proteins may be cloned into any of a number of well known expression plasmids (Sambrook et al., supra) and/or viral vectors, preferably adenoviral or retroviral vectors (see for example, Jacobs et al., J. Virol. 66:2086-2095 (1992), Lowenstein, Bio/Technology 12:1075-1079 (1994) and Berkner, Biotechniques 6:616-624 (1988)), under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hydrogels), and the like, or, in the case of viral-based vectors, by systemic administration. Tissue specific promoters may optionally be employed, assuring that the peptide of interest is expressed only in a particular tissue or cell type of choice. Methods for recombinantly preparing such nucleic acid-based vehicles are well known in the art, as are techniques for administering nucleic acid-based vehicles for protein production.

For the purposes of this invention, the methods of administration are chosen depending on the condition being treated and the particular pharmaceutical composition. Administration of the compositions can be done in a variety of ways, including, but not limited to, cutaneously, subcutaneously, intravenously, orally, topically, transdermally, intraperitoneally, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by intravenous or parenteral injections.

In one preferred embodiment, the method of administration of GGA is by oral delivery, in the form of a powder, tablet, pill, or capsule. Pharmaceutical formulations for oral administration may be made by combining GGA with suitable excipients, such as sugars (e.g., lactose, sucrose, mannitol, or sorbitol), cellulose (e.g., starch, methyl cellulose, hydroxymethyl cellulose, carboxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like.

Alternatively, oral formulations of GGA are in prepared in a suitable diluent. Suitable diluents include various liquid form (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. In some embodiments, lipohilic solvents are used, including oils, for instance, vegetable oils, peanut oil, sesame oil, olive oil, corn oil, safflower oil, soybean oil, etc.; fatty acid esters, such as oleates, triglycerides, etc.; cholesterol derivatives, including cholesterol oleate, cholesterol linoleate, cholesterol myristilate, etc.; liposomes; and the like.

In yet another preferred embodiment, the administration is carried out cutaneously, subcutaneously, intraperitonealy, intramuscularly and intravenously, particularly with regard to the subject gene therapy applications. The pharmaceutical compositions for injection may be prepared in lipophilic solvents, which include, but is not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes. The compositions may be prepared directly in the lipophilic solvent or preferably, as oil/water emulsions, (see for example, Liu, F. et al., Pharm. Res. 12: 1060-1064 (1995); Prankerd, R. J. J. Parent. Sci. Tech. 44: 139-49 (1990); and U.S. Pat. No. 5,651,991).

I Treatment with GGC In an Animal Model Of Glaucoma

Methods

The procedures used in this study were approved by the Animal Research Committee of the University of California, Los Angeles and complied with the ARVO Statement for the Use of Animals in Ophthalmic and Vision Research. Male Wistar rats weighing 250 to 300 g were housed with standard chow and water provided ad libitum. The animal room was lit with fluorescent lights (330 lux) automatically turned on at 6 AM and off at 6 PM, and was maintained at 21° C.

General Scheme

Three experiments are summarized here, and details are provided in the subsequent sections. Experiment 1 was performed to evaluate the expression of HSP72 and HSC70 in RGCs after systemic administration of GGA with Western blot analysis and immunohistochemistry. For Western blotting, twelve rats were equally divided into 6 groups. Three groups of animals were given intraperitoneal injections of GGA 200 mg/kg daily and were euthanized after 1-, 3- or 7 days of administration of GGA. Three control groups were intraperitoneally administered 1) saline-vehicle daily for 7 days; 2) GGA with 4 mg/kg of quercetin (Sigma, St. Louis, Mo.) daily for 7 days; and 3) untreated animals. Enriched RGC fraction was harvested from 2 retinas of each group and used for Western blot analysis. The same experiment for isolation of RGCs and Western blotting was repeated with the other 2 retinas-from each group. For immunohistochemical staining for HSP72 and HSC70, six rats were administered GGA and another 6 rats were given saline systemically for 7 days.

The number of animals used for Experiments 2 and 3 are listed in Table 1:

TABLE 1
Sample size in Experiment 2 and Experiment 3.

| Group | Experiment 2 | Experiment 3 |
|---|---|---|
| Vehicle | 24 | 8 |
| Laser + Vehicle | 24 | 9 |
| Laser + GGA | 23 | 13 |
| Laser + GGA + Q | 8 | 13 |
| GGA | 22 | 6 |
| GGA + Q | 6 | 7 |

GGA, intraperitoneal GGA injection;
Q, intraperitoneal injection of quercetin.

Experiment 2 was performed to investigate whether the induction of HSP72 by GGA enhances RGC survival and protects optic nerve axons in a rat glaucoma model. After pretreatment with GGA (200 mg/kg daily) for 7 days, trabecular laser photocoagulation was performed on one eye of each rat (intracameral injection of India ink was performed 5 days before photocoagulation), while the contralateral eye remained untreated. GGA was then given twice a week at the same dose until euthanasia. Sustained elevation of intraocular pressure (IOP) was maintained by performing trabecular laser photocoagulation three weeks after the first photocoagulation. To elucidate the role of HSP expression in the neuroprotective effects of GGA, systemic administration of quercetin at 4 mg/kg was given in the same manner as GGA. Administrations of saline-vehicle, GGA, or GGA with 4 mg/kg of quercetin without trabecular laser photocoagulation were included as controls. IOP and body weight were measured once a week. After 5 weeks of IOP elevation, the number of retrogradely labeled RGCs with dextran tetramethylrhodamine (DTMR) was counted (n=53). The grading of optic nerve injury and the counting of cresyl violet-stained cells in the retinal ganglion cell layer (RGCL) was also performed (n=54).

Experiment 3 was performed to investigate the inhibition of apoptosis with GGA administration after 1 week of IOP elevation (n=56). TdT-mediated biotin-dUTP nick end labeling (TUNEL) and immunohistochemical analysis for HSP72 and HSC70 were performed.

Administration of GGA

GGA was a gift from Esai Co, Ltd (Tokyo, Japan). A solution of 80 mg/mL GGA was prepared in saline (Balanced salt solution; Alcon Laboratories, Inc., Fort Worth, Tex.) and emulsified for one hour in an ultrasonic generator (Branson Ultrasonic Corp., Danbury, Conn.) immediately before administration. Intraperitoneal injections of GGA were given at a dose of 200 mg/kg. Saline-vehicle was prepared and administered in the same fashion in vehicle-treated control groups.

Isolation of RGCs

A previously described method was modified to partially purify RGCs from other retinal cells in rat retinas.38 Briefly, two dissected rat retinas from each subgroup were washed in 2.5 ml of calcium- and magnesium-free phosphate buffered saline (PBS) at pH 7.4, and incubated in 1.25 ml of PBS containing 0.5 mg/ml trypsin and 0.01% deoxyribonuclease for 15 minutes at 37° C. This was followed by washing the retinas twice in 2.5 ml of minimal essential medium (MEM) containing 10% (vol/vol) fetal bovine serum. The retinas were subsequently washed in 2.5 ml of MEM twice and dissociated in 3 ml of MEM. The cell suspension was then mixed with 1.5 ml of 30% metrizamide (ICN Biomedicals, Inc., Aurora, Ohio) in MEM to a final concentration of 10 metrizamide. This mixture was then overlaid with 5% metrizamide in MEM, and the gradient was centrifuged at 4500 rpm (HB-4; Sorvall Instruments, Newtown, Conn.) for 25 minutes at 4° C. The cells in the 5% to 10% interface were collected. and washed in 25 ml of cold MEM. The washed cells were pelleted by centrifugation at 250×g for 10 minutes (Juan 3000C centrifuge, Winchester, Va.). The cells were then resuspended in 400 μl of MEM buffer, and the protein concentration in the cell suspension was measured with a bicinchoninic acid (BCA) protein assay kit (Pierce, Rockford, Ill.).

Immunoblot

Western blot analysis was performed according to the procedure described by Towbin et al.39 Aliquots of 20 μg of protein from enriched RGCs were separated on a 12% SDS-polyacrylamide minigel (Bio-Rad, Hercules, Calif.) and transferred to the membrane (Immunobilon-P; Millipore Corporation, Bedford, Mass.). The membrane was blocked by incubation in 0.1% Tween-20 in 100 mM Tris-buffered saline containing 10% nonfat dried milk for 1 hour. The membranes were incubated with mouse monoclonal antibody against HSP72 (1:1000; StressGen Biotechnologies Corp., Victoria, British Columbia, Canada) or with rat monoclonal antibody against HSC70 (1:1000; StressGen) overnight and then biotinylated rabbit anti-mouse secondary antibody (1:500; Amersham Pharmacia Biotech, Inc., Piscataway, N.J.) or biotinylated goat anti-rat secondary antibody (1:500; Amersham Pharmacia) for 1 hour. This was followed by incubation with streptavidin-conjugated horseradish peroxidase (1:2000; Amersham Pharmacia) for 40 minutes. The immunoreactive bands were detected by chemiluminescence with an enhanced chemiluminescence Western blot reagent (Amersham Pharmacia).

Immunohistochemistry

Animals were deeply anesthetized with intramuscular injections of 0.8 ml/kg of a cocktail of 5 ml ketamine (100 mg/ml), 2.5 ml xylazine (20 mg/ml), 1.0 ml acepromazine (10 mg/ml), and 1.5 ml normal saline. Then they were transcardially perfused with 4% paraformaldehyde in 0.1 M phosphate buffer. The enucleated eyeballs were immersed in fixative for 1 hour, bisected and post-fixed overnight. The eyes were embedded in paraffin and sectioned at a four-μm thickness along the vertical meridian through the optic nerve head. After deparaffinization and hydration, a species-specific Vectastain ABC kit (Vector Laboratories, Inc., Burlingame, Calif.) was chosen to match the species of primary antibody and the manufacturer's procedures were followed. The tissue sections were incubated with blocking serum solution in PBS for 1 hour. This was followed by incubation with primary antibody at 4° C overnight. The antibodies were mouse monoclonal antibody against HSP72 (1:500; StressGen Biotechnologies Corp., Victoria, British Columbia, Canada), goat polyclonal antibody against HSP72 (1:1000; Santa Cruz Biotechnology, Inc., Santa Cruz, Calif.) or rat monoclonal antibody against HSC70 (1:200; StressGen). Antigen-antibody complexes were detected by an avidin-biotin-peroxidase technique (Vectastain ABC Kit; Vector Laboratories). Diaminobenzidine (DAB) was used to produce a brown color in the target tissue and the slides were permanently mounted. As a negative control, alternate retinal section was incubated with blocking solution by replacing the primary antibody or with anti-rabbit secondary antibody by replacing the original secondary antibody.

Immunohistochemical staining was analyzed quantitatively with a computer-assisted image processing unit (Image-Pro Plus software, Media Cybernetics, Silver Spring, Md.) and the “count-measure” function. Images of immuno-stained sections were captured with a digital camera (Cool snap, RS Photometric, Tucson, Ariz.) attached to the microscope (Axio plan, Carl Zeiss, Oberkochen, Germany) at 630× magnification under oil immersion. The system was calibrated according to the supplier's manual before the analysis. For each digital image, all individual cells in the RGCL were marked by a masked examiner and the optical density of each cell was measured. The relative intensities of cells in the RGCL were measured and averaged (±SEM) to yield a single value representing one retina.

Rat Glaucoma Model

Rats were anesthetized with intramuscular injections of 0.8 ml/kg of the anesthetic cocktail described above. A previously published procedure was modified to produce chronic moderately elevated IOP unilaterally, while the untreated contralateral eye served as the comparative control.40 Animals were injected intracamerally with 10 μl of 35% India ink (Becton Dickinson, Cockeysville, Md.) in 0.01 M PBS after removing a similar volume of aqueous. At the end of the procedure, Tobrex ophthalmic ointment (tobramycin 0.3%; Alcon, Fort Worth, Tex.) was applied topically. Five days after intracameral injection of India ink, a dark band at the limbus was noted due to the aggregation of carbon particles in the trabecular meshwork.38 After anesthesia, approximately 200 laser burns were delivered ab externo to the pigmented trabecular band at laser settings of 200 μm diameter, 200 mW power and 0.2 seconds duration. Three weeks after the first laser treatment, another trabecular laser photocoagulation was performed without further injection of ink.

Measurements of IOP

Dark-phase IOP measurements were monitored once a week with a portable tonometer (Tonopen XL; Mentor O&O, Norwell, Mass.) and were performed one hour after lights off.41 All IOP measurements were performed with animals in the awake state.42 After topical instillation of Alcaine (proparacaine hydrochloride 0.5%; Alcon, Fort Worth, Tex.), the tonometer was gently and briefly applied to the cornea and IOP readings were recorded. Five consecutive readings were taken. The IOP data collected in this study represented as uncorrected Tonopen units. The readings generated by a very light touch or excessive force were ignored. Three readings were obtained by eliminating the minimum and maximum measurements and were averaged.

Evaluation of RGC Density

Rats were euthanized after 5 weeks of IOP elevation to evaluate the number of DTMR (3000 molecular weight, anionic, lysine fixable; Molecular Probes, Eugene, Oreg.) labeled cells, which were considered as surviving RGCs.38 At 48 hours before euthanasia, retrograde labeling was performed in anesthetized animals. The optic nerve was exposed through a lateral conjunctival incision and the optic nerve sheath was incised with a needle knife 2 mm longitudinally starting 3 mm behind the eye. A cross section of the optic nerve was made with the needle knife through the opening of the optic nerve sheath, with care not to damage the adjacent blood supply. DTMR crystals were applied to the proximal cut surface of the optic nerve to label RGCs by fast retrograde axonal transport. After euthanasia and enucleation, the retinas were dissected and flattened with four radial cuts (superotemporal, inferotemporal, superonasal, and inferonasal). They were placed with vitreal side up on glass slides, dried in the dark at room temperature overnight and mounted. The retinas were examined with a fluorescence microscope (Axioplan; Carl Zeiss, Oberkochen, Germany) equipped with a filter that permits visualization of rhodamine fluorescence (excitation filter BP 546, barrier filter LP590; Carl Zeiss). The counting of RGCs was conducted by 2 examiners in a masked fashion. Three areas per retinal quadrant (superior, temporal, inferior and nasal) at 1, 2, and 3 mm from the optic disc were analyzed yielding 12 separate retinal areas for RGC counting. Each rectangular area measured 0.475 mm×0.362 mm and the total counted area corresponded to approximately 3.1% of each total retinal area. Data are expressed as number of RGCs per mm2.

Grading of Optic Nerve Injury and Cell Counting in the Retinal Ganglion Cell Layer (RGCL)

To examine the effect on RGC axons, optic nerve injury was evaluated with an established method.41 After 5 weeks of IOP elevation, deeply anesthetized animals were perfused with a solution of 4% paraformaldehyde and 1% glutaraldehyde. Optic nerve segments 1 rum behind the globe were dissected, washed, postfixed with 5% glutaraldehyde, dehydrated, and embedded. One μm-thick sections were cut and stained with 1% toluidine blue. Optic nerve cross sections were examined under light microscopy and assessed by three independent masked observers. A graded scale of optic nerve injury ranging from 1 (normal) to 5 (total degeneration) was used. Data obtained from three observers were averaged and presented as mean ±SEM.

Corresponding loss of cells from the RGCL was evaluated by counting cells in the RGCL in cresyl violet-stained retinas. After collecting the optic nerves, enucleated eyeballs were postfixed in 10% neutral buffered formalin for 1 hour and washed in 0.1 M phosphate buffer (pH 7.4). The retinas were dissected and flat mounted on a slide, vitreal side up. Four radial cuts were made in the peripheral retinas. The specimens were dried overnight, stained with 1% cresyl violet, dehydrated, and covered with coverslips. Morphologically distinguishable glial cells and vascular endothelial cells were not counted. Cells with cytoplasm rich in Nissl substance and with irregular outlines were counted as neurons.43 The numbers of neurons in the RGCL in regions 1 mm (posterior), 2 mm (mid-peripheral) and 3 mm (peripheral) from the center of the optic nerve head were taken with an eye-piece reticule of a microscope at 400× magnification. The counting was performed by two investigators in a masked fashion and averaged. Results from the four quadrants (superior, temporal, inferior and nasal) of each retina were averaged to give one value (mean ±SEM).

TUNEL Analysis

Four-μm thick paraffin embedded sections along the vertical meridian of the optic nerve head were collected and a minimum of 6 retinal sections (8 μm apart) per eyeball was used for counting the number of TUNEL positive cells in the RGCL. Only full length and undamaged retinal sections without oblique orientation were used. The procedures described in the ApopTag Peroxidase In Situ Apoptosis Detection Kit (Intergen Co., Purchase, N.Y.) were followed and diaminobenzidine (Sigma, St. Louis, Mo.) was used as a color substrate. Counting was performed by two masked investigators with light microscopy, and averaged.

Statistical Analysis

The data are expressed as mean ±SEM. Mean values among groups were compared with oneway ANOVA, and values between groups were compared with the unpaired Student's West. Statistical significance was declared for P<0.05. Two-tailed tests were used for all comparisons.

### EXAMPLE 1

### Induction of HSP72 in RGCs after Administration of GGA

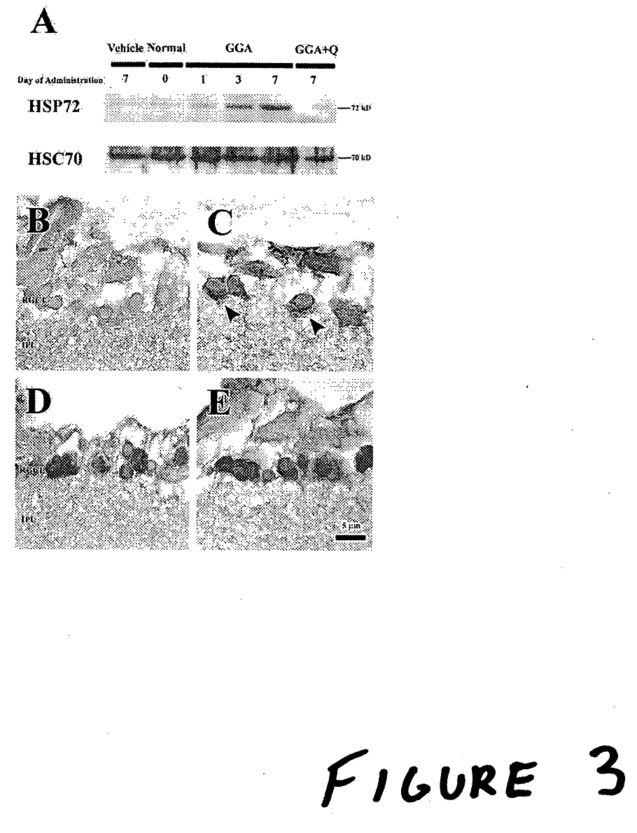

The immunoblots of proteins in the enriched RGC fraction from the rat retinas after systemic administration of GGA (200 mg/kg daily) were probed with antibody against HSP72 (FIG. 3A upper panel) that specifically recognized the inducible form of HSPs as well as antibody against HSC70 (FIG. 3A lower panel) that corresponded to the constitutive form. There was a weak immunoreactivity against HSP72 in RGCs from the vehicle-treated rat retinas (lane 1) and normal untreated control rat retinas (lane 2). One day after administration of GGA, a mild increase in immunoreactivity of HSP72 was noted in RGCs (lane 3). A strong increase in immunoreactivity was detected in RGCs given GGA for 3 and 7 days (lanes 4 and 5 respectively). The expression of HSP72 in RGCs from GGA-treated rats was inhibited by co-administration of quercetin (4 mg/kg; lane 6). However, there was strong immunoreactivity against HSC70 in RGCs of the retinas from control groups (lane 1, 2 and 6) and GGA-treated groups (lane 3 to 5), but there was no detectable difference among them.

To localize the immunoreactivity of inducible and constitutive forms of HSPs in RGCs, immunohistochemical staining for HSP72 and HSC70 was performed on retinal sections after 7 days of GGA administration or vehicle treatment. Increased immunoreactivity of HSP72 was detected in majority of cells in the RGCL after GGA administration (FIG. 3C) when compared with vehicle-treated rat retinas (FIG. 3B). No remarkable change in immunoreactivity of HSP72 was detected in other retinal layers (data not shown). Similar to Western blot analysis, no observable difference in HSC70 expression was noted in the cells in the RGCL (FIG. 3E) or other retinal layers of GGA-treated rats (data not shown) compared with vehicle-treated rats (FIG. 3D).

The foregoing experiments were conducted using intra peritoneal administration of GGA, which demonstrates that GGA is able to cross the blood brain barrier and act on retinal neuronal cells. To confirm that GGA is also effective at inducing HSP72 expression when administered orally, six rats were orally administered a daily dose of GGA for a one week period, each administration being equal to the amount that was administered intra peritoneally in the above experiment. One week after the last oral administration, the retinal cells of the rats were assayed by immuno histochemical staining and also shown to have increased levels of HSP72 in RGCs. These results demonstrated that oral administration of GGA is also effective in inducing expression of HSP72 in retinal cells.

### EXAMPLE 2

### Protection of RGCs by Administration of GGA

The baseline TOP in the awake rats was 15.0±0.6 mmHg as measured by Tonopen (FIG. 4; n=53). Increased TOP was sustained for 5 weeks, with a maximum of 25.6±1.0 mmHg at 4 weeks. The relative increase of IOP at 5 weeks compared with the contralateral eyes was 66% (P=0.001). In the GGA group, the increase of TOP at 5 weeks compared with contralateral control eyes was 82% with a maximum of 27.6±1.2 mmHg. In the group in which quercetin was co-administered with GGA, there was a 59% increase of TOP with a maximum of 25.0±1.7 mmHg compared with the contralateral eye. There were no statistically significant differences between the TOP course of the groups that received vehicle, GGA or GGA and quercetin.

The body weights of rats in the vehicle, GGA, and GGA with quercetin groups were monitored (Table 2). From the first day of saline injection (1 week before the first trabecular laser photocoagulation) to euthanasia (5 weeks after the first laser photocoagulation), the percentage increase of body weight in vehicle-treated rats was 38%, 27% in the GGA group and 38% in the GGA with quercetin group. The gain in body weight among these groups showed no statistically significant difference.

TABLE 2
Time course of body weight in Experiment 2.

| | Weight (g) | | | | | | |
|---|---|---|---|---|---|---|---|
| Group | −1 week | 0 week | 1 week | 2 week | 3 week | 4 week | 5 week |
| Vehicle | 346 ± 7 | 376 ± 8 | 405 ± 10 | 432 ± 11 | 453 ± 12 | 465 ± 11 | 479 ± 12 |
| GGA | 371 ± 6 | 379 ± 8 | 400 ± 9 | 424 ± 9 | 448 ± 11 | 454 ± 12 | 471 ± 12 |
| GGA + Q | 322 ± 4 | 342 ± 7 | 374 ± 8 | 398 ± 9 | 423 ± 10 | 439 ± 11 | 445 ± 12 |

Data are expressed as mean ± SEM.
GGA, intraperitoneal GGA injection;
Q, intraperitoneal quercetin injection. (P = 0.07; ANOVA)

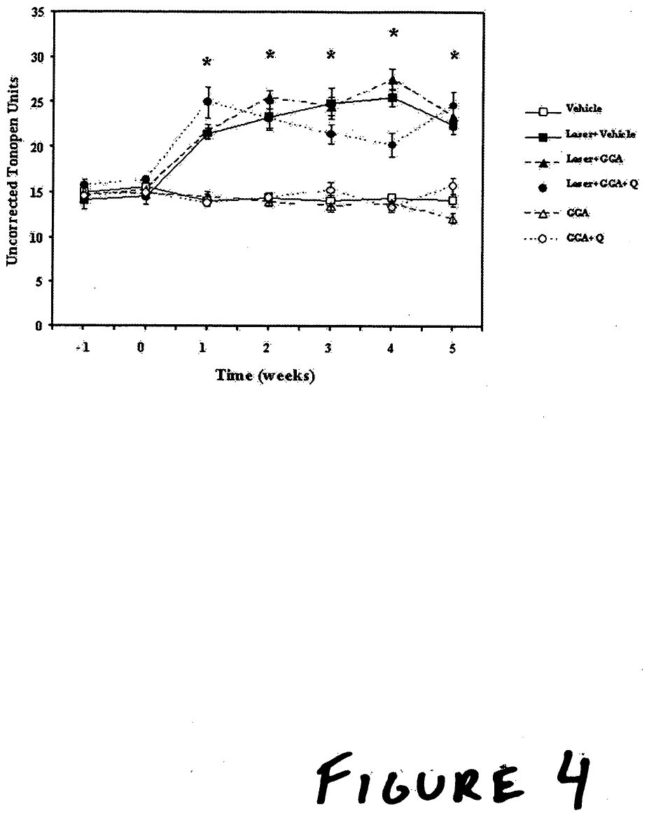

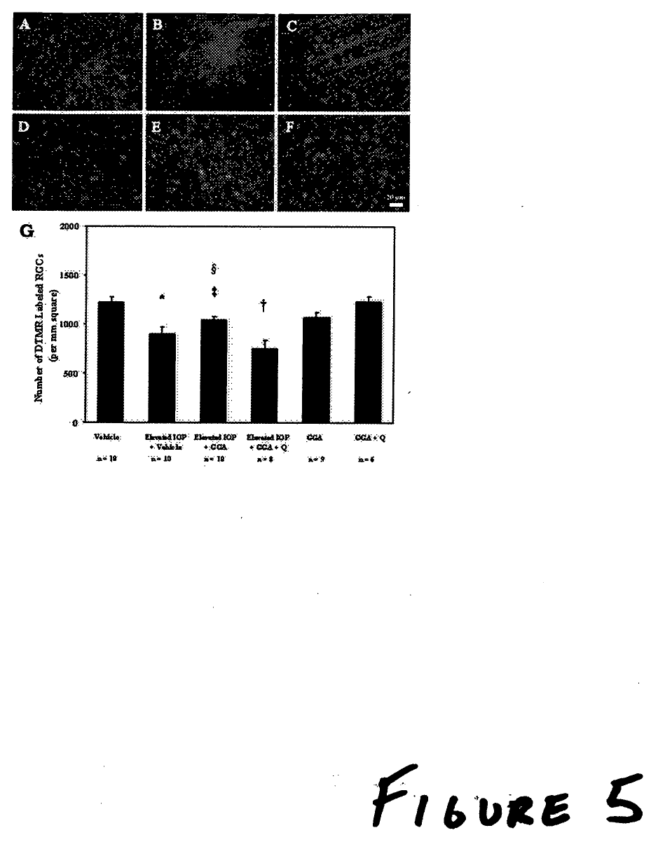

Retrograde labeling with DTMR was performed on optic nerves 2 days before euthanasia to label surviving RGCs by retrograde axoplasmic transport (FIGS. 3A-F). The DTMR-labeled RGCs were counted to evaluate the effect of administration of GGA (FIG. 5G). There was a statistically significant difference between the densities of DTMR-labeled RGCs among the six groups (P=0.001, ANOVA). The density of DTMR-labeled RGCs for vehicle-treated control was 1230 f 51 cells/mm.2 After 5 weeks of TOP elevation, the density of DTMR-labeled RGCs dropped to 904±71 cells/mm2 (FIG. 5B), which corresponded to a 27%±6% reduction when compared to the contralateral eyes (P=0.0003). Administration of GGA preserved 57% more DTMR-labeled cells (1044±36 cells/mm2, FIG. 5C) compared with vehicle. The preservation of RGCs by administration of GGA in retinas with IOP elevation was partial (P=0.003 when compared with vehicle-treated controls). Co-administration of quercetin abolished the protective effect of GGA in the retinas with IOP elevation (FIG. 5D; P=0.002), which showed a density of 756±88 cells/mm.2 The density of DTMR-labeled RGCs in GGA-treated contralateral controls (FIG. 5E), and GGA and quercetin-treated contralateral controls (FIG. 5F) was 1077±48 cells/mm2 and 1235±51 cells/mm2, respectively. There was no statistical significance between the densities of DTMR-labeled RGCs in GGA-treated controls and vehicle-treated controls (P=0.08) and between GGA and quercetin-treated controls and vehicle-treated controls (P=0.1).

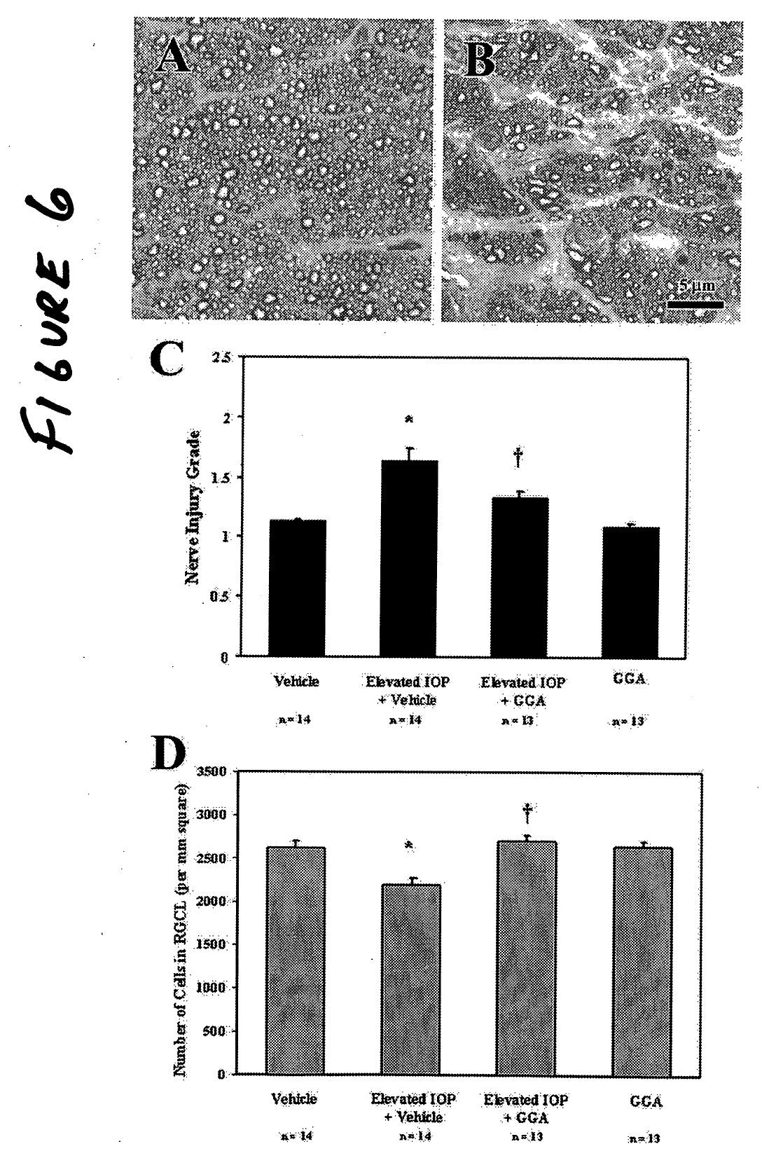

Axonal injury in the optic nerve was demonstrated by light microscopy (FIGS. 6A & B) and graded from 1 (no nerve injury) to 5 (severe nerve injury). A normal optic nerve with a grade of 1 is shown in FIG. 4A while an optic nerve with a grade 2 injury is shown in FIG. 4B. There was significant damage to the optic nerve after 5 weeks of sustained IOP, with a grade of 1.64±0.10 compared with contralateral controls (1.13±0.02, P=0.001), indicating mild to moderate injury. The optic nerve injury was significantly ameliorated by the administration of GGA, with a grade of 1.33±0.05 (P=0.026). The GGA-treated contralateral control eyes showed no statistically significant optic nerve injury (1.11±0.02).

Cresyl violet staining and cell counting revealed a significant reduction of cells in the RGCL (2193±75 cells/mm2 corresponding to 16% loss) in eyes after 5 weeks of elevated IOP when compared with contralateral eyes (2620±78 cells/mm2; P=0.001) as shown in FIG. 4D. Administration of GGA inhibited the loss of cells in the RGCL with IOP elevation (2697±70 cells/mm2, P=0.001) and had no significant effect on the number of cells in the RGCL of GGA-treated contralateral control retinas (2644±59 cells/mm2).

### EXAMPLE 3

### Inhibition of Cell Death by GGA

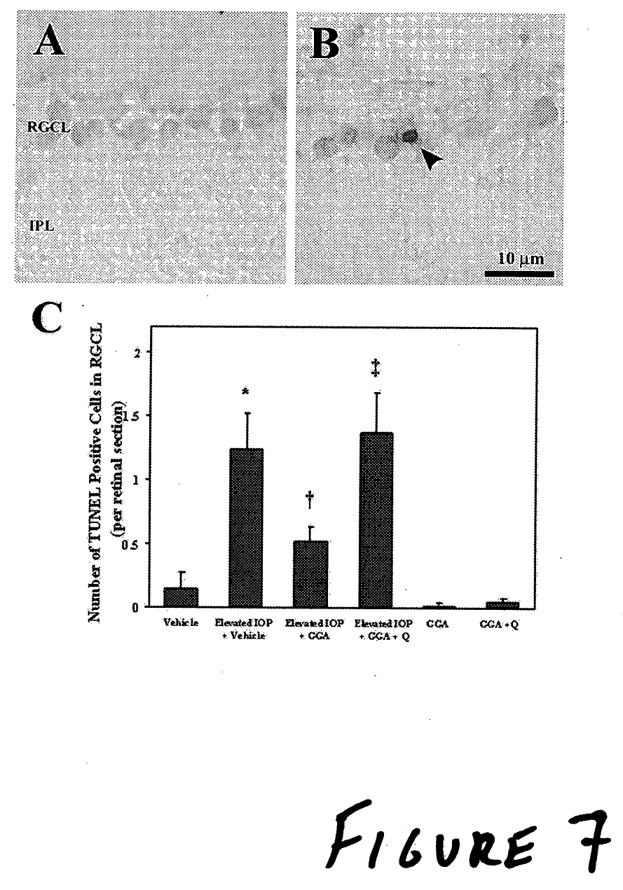

TUNEL staining was performed to label dying cells (FIG. 7B is shown as representative) in retinas with elevated IOP. The number of TUNEL positive cells in the RGCL were counted and compared to evaluate the effect of GGA (FIG. 5C). After 1 week of TOP elevation, the number of TUNEL positive cells in the RGCL was 1.24±0.29 per retinal section and was statistically significantly higher than the control groups treated with vehicle (P=0.026), GGA (P=0.008) or GGA with quercetin (P=0.017). The administration of GGA significantly reduced the number of TUNEL positive cells to 0.53±0.11 per retinal section. (P=0.02), corresponding to a 57% inhibition of cell death after 1 week of IOP elevation. The number of TUNEL positive cells of quercetin-treated retinas with IOP elevation and GGA administration was 1.37±0.31 per retinal section, similar to the vehicle-treated retinas with IOP elevation.

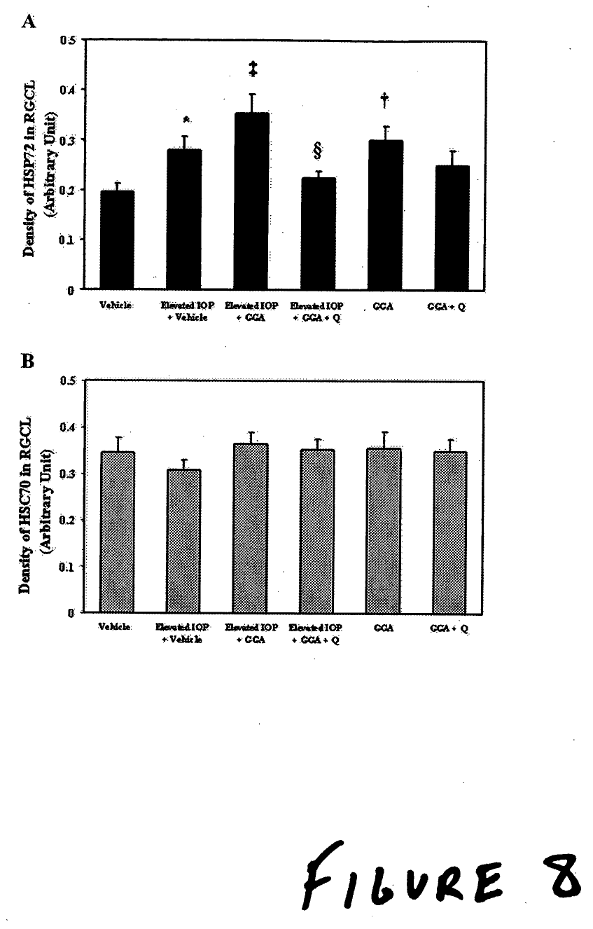

Quantitative analysis of immunoreactive intensity of HSP72 (FIG. 8A) and HSC70 (FIG. 8B) in the RGCL was performed 1 week after trabecular laser photocoagulation. The expression of HSP72 immunoreactivity was a statistically significantly different among the groups (P=0.001, ANOVA). There was a statistically significantly increased expression of HSP72 induced by IOP elevation (P=0.01). HSP72 expression in retinas with IOP elevation apparently further increased after GGA administration (P=<0.001 when compared with vehicle control) but this increase was not statistically significant when compared with the retinas with IOP elevation alone. HSP72 expression in retinas with IOP elevation and GGA administration was significantly reduced by the co-administration of quercetin with the retinas with IOP elevation (P=0.002). Systemic administration of GGA alone caused an increased expression of HSP72 in the RGCL when compared with vehicle-alone controls (P=0.005) but this increase was abolished by co-administration of quercetin. In contrast, there was no statistically significant difference in the expression of HSC70 in RGCL among all the groups.

### Increasing Expression of HSP70 by Gene Delivery

The inventors have recognized that RGCs in glaucoma undergo apoptosis, although the molecular pathways of this process are not completely understood. Accordingly, another aspect of the invention is treatment of glaucoma is preserving RGCs via over-expression of anti-apoptotic genes such as HSP 70. While not being bound by theory, the HSP70 neuroprotective effect may be explained by its ability to block the assembly of functional apoptosomes. The binding of HSP70 to Apaf-1 prevents recruitment of caspases to the apoptosome complex. Moreover, HSP70 could inhibit caspase-independent cell death by interacting with the apoptosis inducing factor (AIF). Although HSP expression is induced endogenously in response to stress, the level of the protein in injured cells may not be sufficient to have a protective effect.

A number of studies have been published where HSP70 has been overexpressed in various neuronal and non-neuronal cell lines, yielding protection against numerous insults, including heat shock, oxidative stress, apoptotic stimuli, and ischemia-like conditions. HSP70 gene therapy using HSV vectors has been shown to produce a neuroprotective effect in rat models of stroke and epilepsy when delivered before or after insult (see Yenari et al. Neurol Res. 23(5):543-52 (2001); also Hoehn et al. J Cereb Blood Flow Metab. 21(11):1303-9 (2001). Furthermore, induction of HSP70 expression has been demonstrated to reduce RGC degeneration in a rat glaucoma model (see Park et al., supra.

The present invention accordingly also contemplates use of HSP70 protein gene therapy to protect retinal neurons from retinal degeneration suitable for use in vivo in a variety of animal systems. HSP70 gene therapy can be a successful therapeutic strategy for treatment of many ocular diseases, such as glaucoma and slowly progressing retinal degenerations, which have complex pathology involving multiple genetic as well as environmental factors.

In a preferred embodiment, the methods comprise contacting neuronal retinal cells with a nucleic acid molecule that functions to increase HSP70 expression in the retinal cells of the subject, whereby the retinal cells are protected from degeneration relative to retinal cells not contacted with the nucleic acid molecule. In certain embodiments, the nucleic acid molecules that function to increase HSP70 expression will be vector nucleic acid molecules operably configured with a sequence that encodes a HSP70 polypeptide that exhibits the neuroprotective effect associated with the HSP70 protein encoded by SEQ ID NO: 1 or 2. In other embodiments, the nucleic acid molecule that functions to increase HSP70 expression in the retinal cells will be a nucleic acid operably configured to express a sequence that encodes transcription factor HSF-1 in the retinal cell, which in turn induces the expression of endogenously encoded HSP70.

By “nucleic acid molecules that encode HSP70,” and grammatical equivalents thereof is meant the nucleotide sequences according to SEQ ID NO: 1 or 2, nucleotide sequences encoding any of the HSP70 family of heat shock proteins Hspa1, Hspa4, Hspa5, HSpt70 and Hspa8 identified hereinabove, as well as nucleotide sequences encoding a polypeptide having at least about 80% sequence identity, usually at least about 85% sequence identity, preferably at least about 90% sequence identity, more preferably at least about 95% sequence identity and most preferably at least about 98% sequence identity to the polypeptide encoded by SEQ ID NO: 1 or 2, any of which when expressed in a retinal cell, exhibits protection against degeneration of retinal neuronal cells.

HSP70 proteins having less than 100% sequence identity with the polypeptide encoded by SEQ ID NO: 2 will generally be produced from native HSP70 sequences from species other than human and variants of native HSP70 nucleotide sequences from human or non-human sources. In this regard, it is noted that many techniques are well known in the art and may be routinely employed to produce nucleotide sequence variants of native HSP70 sequences and assaying the polypeptide products of those variants for the ability to protect against neuroma; degeneration that is characteristic of the HSP70 polypeptides encoded by SEQ ID NO: 1 or 2.

As used herein and further defined below, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequences depicted in FIGS. 1 and 2 also include the complement of the sequence. By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention.

In one embodiment, the present invention provides nucleic acids encoding HSP70 variants. These variants fall into one or more of three classes: substitutional, insertional or deletional variants. These variants ordinarily are prepared by site specific mutagenesis of nucleotides in the nucleotides of the nucleic acid according to SEQ ID NO: 1 or 2, using cassette or PCR mutagenesis or other techniques well known in the art, to produce DNA encoding the variant, and thereafter expressing the DNA in a retinal neuronal cell, as described below, or a recombinant cell culture as outlined herein. Amino acid sequence variants are characterized by the predetermined nature of the variation, a feature that sets them apart from naturally occurring allelic or interspecies variation of the HSP70 amino acid sequence. The variants typically exhibit the same qualitative biological activity as the naturally occurring analogue, although variants can also be selected which have modified characteristics as will be more fully outlined below.

While the site or region for introducing a sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Another example of a technique for making variants is the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each of which is incorporated by reference herein in its entirety. Screening of the mutants is done using assays of heme oxygenase activities, as described above.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the heme oxygenase-I are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The variants typically exhibit the same qualitative biological activity and will elicit the same immune response as the naturally occurring analogue, although variants also are selected to modify the characteristics of the heme oxygenase-I as needed. Alternatively, the variant may be designed such that the biological activity of the protein is altered.

To express HSP70 protein to test for HSP70 activity, a nucleic acid encoding the HSP70 protein is cloned and expressed as outlined below. Probe or degenerate polymerase chain reaction (PCR) primer sequences may be used to find other nucleic acid sequence encoding HSP70 polypeptides from humans or other organisms. As will be appreciated by those in the art, particularly useful probe and/or PCR primer sequences include the unique areas of the nucleic acid sequence according to SEQ.ID Nos. 1 or 2. As is generally known in the art, preferred PCR primers are from about 15 to about 35 nucleotides in length, with from about 20 to about 30 being preferred, and may contain inosine as needed. The conditions for the PCR reaction are well known in the art. It is therefore also understood that provided along with the sequences provided herein are portions of those sequences, wherein unique portions of 15 nucleotides or more are particularly preferred. The skilled artisan can routinely synthesize or cut a nucleotide sequence to the desired length.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins.

Using the nucleic acids of the present invention which encode a protein, a variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA encoding a rough endoplasmic reticulum (RER) resident sequence such as HSP70 is operably linked to DNA encoding a RER transit peptide if the nucleic acid encoding the transit peptide is fused in frame to the sequence encoding the HSP70 polypeptide. A promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the HSP70 protein; for example, transcriptional and translational regulatory nucleic acid sequences from AAV vectors are preferably used to express the HSP70 protein in neuronal cells. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

Another preferred expression vector system is a retroviral vector system such as is generally described in WO 97/27212 and WO 97/27213, both of which are hereby expressly incorporated by reference.

Nucleic acid molecules encoding HSP70 as well as any nucleic acid molecule derived from either the coding or non-coding strand of a nucleic acid molecule that encodes HSP70 may be contacted with retinal cells in a variety of ways which are known and routinely employed in the art, wherein the contacting may be ex vivo or in vivo. The particular protocol will depend upon the nature of the organ, the form of the nucleic acid, and the use of immunosuppressants or other drugs.

By the term “conditions permissive for the contacting of exogenous nucleic acid”, and grammatical equivalents herein is meant conditions which allow cells of the ex vivo or in vivo tissue to be contacted with the exogenous nucleic acid, whereby HSP70 expression is modified. In a preferred embodiment, contacting results in the uptake of the nucleic acid into the cells.

In a preferred embodiment, the nucleic acid encodes a protein which is expressed. In some embodiments, the expression of the exogenous nucleic acid is transient; that is, the exogenous protein is expressed for a limited time. In other embodiments, the expression is permanent

In some embodiments, the exogenous nucleic acid is incorporated into the genome of the target cell; for example, retroviral vectors integrate into the genome of the host cell. Generally this is done when longer or permanent expression is desired. In other embodiments, the exogenous nucleic acid does not incorporate into the genome of the target cell but rather exists autonomously in the cell; for example, many such plasmids are known. This embodiment may be preferable when transient expression is desired.

The permissive conditions will depend on the form of the exogenous nucleic acid. The production of various expression vectors is described above. Thus, for example, when the exogenous nucleic acid is in the form of an adenoviral, retroviral, or adeno-associated viral vector (AAV), the permissive conditions are those which allow viral contact and/or infection of the cell. Similarly, when the exogenous nucleic acid is in the form of a plasmid, the permissive conditions allow the plasmid to contact or enter the cell. Thus, the form of the exogenous nucleic acid and the conditions which are permissive for contacting are correlated. These conditions are generally well known in the art.

Permissive conditions depend on the expression vector to be used, the amount of expression desired and the target cell. Generally, conditions which allow in vitro uptake of exogenous cells work for ex vivo and in vivo cells.

Permissive conditions are analyzed using well-known techniques in the art. For example, the expression of exogenous nucleic acid may be assayed by detecting the presence of mRNA, using Northern hybridization, or protein, using antibodies or biological function assays.

Specific conditions for the uptake of exogenous nucleic acid are well known in the art. They include, but are not limited to, retroviral infection, adenoviral infection, transformation with plasmids, transformation with liposomes containing exogenous nucleic acid, biolistic nucleic acid delivery (i.e., loading the nucleic acid onto gold or other metal particles and shooting or injecting into the cells), adeno-associated virus infection, HIV virus infection and Epstein-Barr virus infection. These may all be considered “expression vectors” for the purposes of the invention.

The expression vectors may be either extrachromosomal vectors or vectors which integrate into a host genome as outlined above. Generally, these expression vectors include transcriptional and translational regulatory nucleic acid operably linked to the exogenous nucleic acid. “Operably linked” in this context means that the transcriptional and translational regulatory DNA is positioned relative to the coding sequence of the exogenous protein in such a manner that transcription is initiated. Generally, this will mean that the promoter and transcriptional initiation or start sequences are positioned 5′ to the exogenous protein coding region. The transcriptional and translational regulatory nucleic acid will generally be appropriate to the host cell in which the exogenous protein is expressed; for example, transcriptional and translational regulatory nucleic acid sequences from mammalian cells, and particularly humans, are preferably used to express the exogenous protein in mammals and humans. Numerous types of appropriate expression vectors, and suitable regulatory sequences are known in the art.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive, tissue specific or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In a preferred embodiment, AAV vectors are used for the delivery of the HSP70 gene to target cells. Recombinant AAV vectors have been used for gene delivery to various eye cell types: RPE, photoreceptors, Muller cells, RGCs, and TM cells. Recombinant AAV vectors are non-pathogenic, lacking significant toxicity or immune response. Recombinant AAV can infect both dividing and non-dividing cells, and allow for long-term transgene expression. The HSP70 AAV vector may be constructed, for example, by cloning of the full length HSP70 cDNA into pKm201CMV (see Lau D, McGee L H, Zhou S, Rendahl K G, Manning W C, Escobedo J A, Flannery J G. Retinal degeneration is slowed in transgenic rats by AAV-mediated delivery of FGF-2. Invest Ophthalmol Vis Sci. 2000 October;41(11):3622-33.). Expression of HSP70 in this vector is driven by the CMV immediate-early promoter/enhancer element. Intravitreal or subretinal injections will be performed to transfect RGCs or photoreceptors and RPE, respectively. Expression of HSP70 will be evaluated by RT-PCR and immunohistochemistry. The cytoprotective effect of HSP70 overexpression will be assessed at morphological and physiological levels. The thickness of the RGC layer and ONL will be compared between treated and untreated glaucoma and retinal degeneration animal models, respectively. ERG recordings will be performed to determine the correlation between physiological function and morphological rescue.

Other preferred embodiments include use of retroviral vectors. Suitable retroviral vectors include but are not limited to LNL6, LXSN, and LNCX (see Byun et al., Gene Ther. 3(9):780-8 (1996) for review).

In other preferred embodiments, adenovirus virus vectors are used. Suitable adenoviral vectors include modifications of human adenoviruses such as Ad2 or Ad5, wherein genetic elements necessary for the virus to replicate in vivo have been removed; e.g., the E1 region, and an expression cassette coding for the exogenous gene of interest inserted into the adenoviral genome (for example AdGVCFTR10).

In other embodiments of the present invention, the nucleic acid molecule is introduced into cells of retinal by liposome-mediated nucleic acid transfer. In this regard, many liposome-based reagents are well known in the art, are commercially available and may be routinely employed for introducing a nucleic acid molecule into cells. Certain embodiments of the present invention will employ cationic lipid transfer vehicles such as Lipofectamine or Lipofectin (Life Technologies), dioleoylphosphatidylethanolamine (DOPE) together with a cationic cholesterol derivative (DC cholesterol), N[1-(2,3-dioleyloxy)propyl]-N,N,N-trimethylammonium chloride (DOTMass.) (Sioud et al., J. Mol. Biol. 242:831-835 (1991)), DOSPA:DOPE, DOTAP, DMRIE:cholesterol, DDAB:DOPE, and the like. Production of liposome encapsulated nucleic acid is well known in the art and typically involves the combination of lipid and nucleic acid in a ratio of about 1:1.

In vivo delivery includes, but is not limited to direct injection into the retina or by other means of perfusion. The nucleic acid and/or delivery vehicle may be administered intravascularly at a proximal location to the retina or administered systemically. One of ordinary skill in the art will recognize the advantages and disadvantages of each mode of delivery. For instance, direct injection may produce the greatest titer of nucleic acid in the retina, but distribution of the nucleic acid will likely be uneven throughout the retinal tissue. Introduction of the nucleic acid proximal to the retina will generally result in greater contact with the cells of the retina, but systemic administration is generally much simpler. The nucleic acids may be introduced in a single administration, or several administrations, beginning before removal of the organ from the donor as well as after transplantation. The skilled artisan will be able to determine a satisfactory means of delivery and delivery regimen without undue experimentation.

In a preferred embodiment, the nucleic acid is contacted with cells of the retinal by direct injection into the retina. In this regard, it is well known in the art that living cells are capable of internalizing and incorporating exogenous nucleic acid molecule with which the cells come in contact. That nucleic acid may then be expressed by the cell that has incorporated it into its nucleus. In an alternate preferred embodiment, the nucleic acid is contacted with cells of the retina by systemic administration.

The above described nucleic acid molecules will function to modulate the overall HSP70 activity of a cell with which it is contacted. In cases where the nucleic acid molecule encodes a polypeptide having at least one activity normally associated with the HSP70 polypeptide, the modulation will generally be exemplified by an increase in the expression of HSP70 in the retinal cell.

From the foregoing it will be appreciated that, although specific embodiments of the invention have been described herein for purposes of illustration, various modifications may be made without deviating from the spirit and scope of the invention. Accordingly, the invention is not limited except as by the following claims.

1. A method of inhibiting degeneration of a neuronal cell in a patient comprising;
administering to the subject an amount of GGA sufficient to induce expression of a HSP70 protein in the neuronal cell.

2. The method of claim 1 wherein said HSP70 protein is HSP72.

3. The method of claim 1 wherein said GGA is administered intravenously.

4. The method of claim 1 wherein said GGA is administered orally.

5. The method of claim 1 wherein the neuronal cell is a retinal neuronal cell.

6. The method of claim 5 wherein the retinal neuronal cell is a RGC.

7. The method of claim 6 wherein the neuronal degeneration is associated with glaucoma.

8. The method of claim 1 wherein the neuronal degeneration is associated with ischemic degeneration of retinal cells.

9. The method of claim 1 wherein the neuronal degeneration is associated with macular degeneration.

10. A method of treating glaucoma in a subject comprising;
administering to the subject having glaucoma an amount of GGA sufficient to induce expression of a HSP70 protein in a retinal ganglion cell of the subject.

11. The method of claim 1 wherein said HSP70 protein is HSP72.

12. The method of claim 10 wherein the GGA is systemically administered.

13. The method of claim 10 wherein the GGA is orally administered.

14. A method of treating a subject to protect against degeneration of a neuronal cell of the retina, comprising;
administering to the subject a nucleic acid operably configured to express in the neuronal cell of the retina, a selected nucleic acid sequence encoding a protein selected from the group consisting of a HSP70 protein, and a protein that induces expression of an endogenous HSP70 gene in the subject.

15. The method of claim 14 wherein the selected nucleic acid sequence encodes HSP72.

16. The method of claim 14 wherein the selected nucleic acid sequence encodes transcription factor HSF-1.


SEQUENCE LISTING (4 sequences)

SEQ ID NO: 1 - DNA, 2073 bp, Rattus norvegicus
  CDS (52)..(1992)
gtctctgtgtggtctcgtcatcagcacagctgggcctacacgcaagcaaccatgtctaagggacctgcggttggcattgatcttggcaccacctactcctgtgtgggtgtcttccagcatggaaaggtggaaataattgccaatgaccagggtaaccgcaccacgccgagctatgttgctttcaccgacacagaacgattaattggggatgcggccaagaatcaggttgcaatgaaccccaccaacacagtttttgatgccaaacgtctgatcggacgtaggttcgatgatgctgttgttcagtctgacatgaagcactggcccttcatggtggtgaacgatgcaggcaggcccaaggtccaagtcgaatacaaaggggagacaaaaagtttctatcctgaggaagtgtcttcaatggttctgacaaaaatgaaggaaattgcagaagcttaccttggaaagactgttaccaatgccgtggtcaccgtgccagcttacttcaatgactctcagcgacaggcaacaaaagatgctggaactattgctggcctcaacgtacttcgaattatcaatgagccaactgctgctgctattgcctatggcttagataagaaggtcggggctgaaaggaatgtgctcatttttgacttgggaggtggcacttttgatgtgtcaatcctcactatcgaggatggaatttttgaagtcaaatcaacagctggagacacccacttgggcggagaagactttgacaaccgaatggtcaaccatttcattgctgagtttaagcgaaagcacaagaaggacatcagtgagaacaagagagctgtcaggcgtctccgcactgcctgtgagcgggccaagcgcaccctctcctccagcacccaagccagtattgagattgattctctctatgagggaattgacttctacacctccattacccgtgctcgatttgaggagttgaatgctgacctgttccgtggcacactggaccctgtagagaaggcccttcgagatgccaaactagacaagtcacagatccatgatattgtcctggtgggtggttctaccagaatccccaagatccagaaacttctgcaagacttcttcaatggaaaagagctgaataagagcattaaccccgatgaagctgttgcctatggtgcagctgtccaggcagccattctatctggagacaagtctgagaatgttcaggatttgctgctcttggatgtcactcctctttcccttgggattgaaactgctggtggagtcatgactgtcctcatcaagcgcaataccaccattcccaccaagcagacccagactttcaccacctactctgacaaccagccaggtgtactcatccaggtgtatgaaggtgaaagggccatgaccaaggacaacaacctgcttgggaagtttgagctcacaggcatacctccagcaccccgtggggttcctcagattgaggttacttttgacattgatgccaatggcatcctcaatgtttctgctgtagataagagcacaggaaaggagaacaagatcaccatcaccaatgacaagggccgcttgagtaaggaggatattgagcgcatggtccaagaagctgagaagtacaaagctgaggatgagaagcagagagataaggtttcctctaagaactcgctggagtcttatgctttcaacatgaaagcaactgttgaggatgagaaacttcaaggcaagatcaatgatgaagacaaacagaagattcttgacaagtgcaacgaaatcatcagctggctggataagaaccagactgcggagaaggaagaatttgagcatcagcagaaagaactggagaaggtctgcaaccctatcatcaccaagctgtaccagagtgctggtggcatgcctggaggaatgcctggtggcttccctggtggaggagctcctccatctggtggtgcttcttcaggccccaccattgaagaggtcgattaagtcaaagtagagggtatagcattgttccacagggacccaaaacaagtaacatggaataataaaactatttaaattggcacc

SEQ ID NO: 2 - protein, 646 aa, Rattus norvegicus
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIISWLDKNQTAEKEEFEHQQKELEKVCNPIITKLYQSAGGMPGGMPGGFPGGGAPPSGGASSGPTIEEVD

SEQ ID NO: 3 - DNA, 2259 bp, Homo sapiens
  CDS (59)..(1999)
ctcttgggttttttgtggcttccttcgttattggagccaggcctacacgccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgccgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccataaacaaaaaaaaaaaaaaa

SEQ ID NO: 4 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD